(19) United States
(12) Patent Application Publication
(10) Pub. No.: US 2012/0171755 A1
(43) Pub. Date: Jul. 5, 2012

(54) FABRICATION OF HOLLOW NANONEEDLES

(75) Inventors: Elad Peer, Haifa (IL); Uri Sivan, Haifa (IL)

(73) Assignee: Technion Research and Development Foundation Ltd., Haifa (IL)

(21) Appl. No.: 13/338,831

(22) Filed: Dec. 28, 2011

Related U.S. Application Data
(60) Provisional application No. 61/429,201, filed on Jan. 3, 2011.

(51) Int. Cl. C12M 1/00

(52) U.S. Cl. 435/283.1; 216/56

(57) ABSTRACT

The present invention provides a method of fabricating a device having hollow nanoneedles which extend through a supporting membrane. The device can be used as a molecular delivery system.

5

### CROSS REFERENCE TO RELATED APPLICATION

The present application claims the benefit of U.S. Provisional patent application Ser. No. 61/429,201 filed Jan. 3, 2011.

### FIELD OF THE INVENTION

The present invention relates to a method of fabricating a nanoneedle or an array of nanoneedles each having an internal aperture which traverses a supporting membrane and use thereof as molecular delivery systems.

### BACKGROUND OF THE INVENTION

Recent research in the field of cellular biology is focused on the delivery of molecules into cell cytoplasms. Molecular delivery can be applied, inter alia, for the insertion of probes and cargo (e.g. drugs) into cells. It was shown to be feasible using hollow or solid needle-like geometries. Methods that have been used for molecular delivery include atomic force microscope (AFM) with modified tips which were shown to puncture single cells followed by the insertion of substances directly into the cytoplasm (Vakarelski et al., Langmuir, 2007, 23: p. 10893). Other methods include the integration of standard microinjection tools with micro fluidic systems (Adamo and Jensen, Lab on a Chip, 2008, 8(8): p. 1258), and the loading of cargo into hollow nanostructures (Peckys et al., Nanotechnology, 2009, 20(14): p. 8) for penetrating cells and subsequently releasing the cargo into the cytoplasm. These methods, however, fail to address multiple cells in culture in a reproducible manner.

Kim et al. (J. Am. Chem. Soc., 2007, 129(23): p. 7228) demonstrated the direct interface of vertically aligned silicon nanowires with mammalian cells. Mann et al. (Acs Nano, 2008, 2(1): p. 69) designed a tetracycline-inducible small hairpin RNA (shRNA) vector system for silencing cyan fluorescent protein (CFP) expression. The vector system was delivered alongside the yfp marker gene into Chinese hamster ovary cells using impalefection on spatially indexed vertically aligned carbon nanofiber arrays (VACNFs). Peckys et al. (Nanotechnology, 2009, 20(14): p. 8) disclosed the modification of gold-coated nanofiber arrays with self-assembled monolayers to which reporter dsDNA was covalently and end-specifically bound with or without a cleavable linker. The DNA-modified nanofiber arrays were then used to impale, and thereby transfect, Chinese hamster lung epithelial cells. Shalek, et al. (PNAS USA, 2010, 107(5): p. 1870) demonstrated the delivery of biomolecules into immortalized and primary mammalian cells using surface-modified vertical silicon nanowires.

The fabrication of high aspect ratio hollow devices which can be used for the delivery of molecules into cells remains a challenge. Most conventional microfabrication techniques result in devices that contain hollow structures (e.g. hollow nanotube arrays) which are attached to a supporting substrate. The hollow structures can be formed by physical milling or by using selective chemical etching of non-hollow structures, or by growing structures with hollow shapes (nanotubes, etc.) on the supporting substrate. However, the inner hole of the devices does not traverse the supporting substrate.

Existing methods for perforating substrates are either slow, expensive, cause chemical and physical damage to the substrates (e.g., FIB milling), or suffer from poor alignment of the devices and from lack of flexibility in mixing devices of different shapes and sizes on the same substrate. In addition, the aspect ratio of supported hollow devices in the nanometric scale is limited due to technical problems and the fragility of devices at these small sizes. Other molecular delivery systems include hollow nanotubes which are not supported by a sold substrate. Recently, Skold, et al., (Nanotechnology, 21(15): p. 155301) disclosed a scheme for producing nanotube membranes using free-standing hollow nanowires having GaAs—AlInP core-shell. The nanowires were grown by metal-organic vapor phase epitaxy and were partially embedded in a polymer film. The GaAs core and substrate were etched selectively, leaving tubes with open access to both sides of the membrane. Electrophoretic transport of T4-phage DNA through the hollow nanowires was demonstrated using epifluorescence microscopy.

There is an unmet need for a method of fabricating hollow nanoneedles or nanotubes with holes that traverse a supporting membrane for delivering molecules into cell cytoplasms.

### SUMMARY OF THE INVENTION

The present invention provides a method of fabricating a device comprising a hollow nanoneedle or an array of hollow nanoneedles which are supported by a membrane wherein the hollow interior aperture of the nanoneedle penetrates the membrane and spans the membrane from one side to the other. The method comprises the perforation of a membrane which serves as a substrate, followed by coating of the perforated substrate and the etching of the coating from one side of the membrane followed by the partial etching of the membrane from the same side in a selective manner.

The present invention is based in part on the unexpected finding that hollow nanostructures with holes that traverse a supporting membrane can be produced by defining holes and coating them with a material which allows selective etching. Unexpectedly, when selectively etching the supporting membrane, devices with self-aligned holes, high aspect ratio and high throughput are obtained. The method of the present invention provides structures having different sizes and shapes, thus enabling the combination of different nanostructures on the same substrate. Furthermore, the method of the present invention is not limited to a particular material and can be applied to any pair of materials (coating and membrane) that can be etched selectively due to differences in the rate of etching or by using different etching reagents.

According to a first aspect, the present invention provides a method of fabricating a device comprising at least one hollow nanoneedle, wherein the nanoneedle is supported by a membrane having two opposing surfaces and wherein the opening or aperture in the nanoneedle traverses the membrane, the method comprising the steps of:

a. perforating a membrane to form at least one hole that spans the membrane from one side to the opposite side;

b. coating the surfaces of the perforated membrane obtained in step (a) with a material that is distinct from the material forming the membrane;

c. removing the coated surface obtained in step (b) from one side of the membrane; and

d. selectively etching a portion of the membrane from said one side of the membrane.

It will be recognized by one of skill in the art that the selective etching is performed such that the membrane is etched at a higher rate than the coating of the membrane. Other selective etching comprises the use of a reagent to which only the membrane is susceptible.

In one embodiment, the method of the present invention provides the fabrication of a device for delivering molecules into cells.

In another embodiment, the membrane is selected from the group consisting of silicon, silicon dioxide, silicon nitride, germanium, germanium dioxide, germanium nitride, aluminum, alumina, boron nitride and combinations thereof Each possibility represents a separate embodiment of the present invention.

In particular embodiments, the membrane is selected from the group consisting of silicon, silicon dioxide, silicon nitride and combinations thereof. In yet another embodiment, the membrane comprises silicon. Each possibility represents a separate embodiment of the present invention.

In some embodiments, the coating of the surfaces of the membrane is selected from the group consisting of silicon dioxide, silicon nitride, germanium dioxide, germanium nitride, and alumina. Each possibility represents a separate embodiment of the present invention.

In various embodiments, the surface of the membrane is selected from the group consisting of silicon dioxide and silicon nitride. Each possibility represents a separate embodiment of the present invention.

It will be appreciated by one of skill in the art that the coating may be obtained by applying an external layer to the surfaces or by exposing the surfaces to chemical alterations or modifications. Non-limiting examples of chemical alterations that may be used to produce a coating include but not limited to oxidation or nitridation.

In alternative embodiments, it will be appreciated by one of skill in the art that the surface coating may be formed or deposited on the perforated membrane in such a manner that one of the opposing surfaces remains uncoated. For example, the coating of one surface may be prevented by masking of one side of the membrane. In this case, the removal of the coating from one side of the membrane according to step (c) is not required prior to the selective etching that creates the hollow nanoneedles.

In other embodiments, the step of perforating at least one hole in a membrane comprises the use of at least one technique selected from plasma and Focused Ion Beam (FIB). Each possibility represents a separate embodiment of the present invention. In one embodiment, the step of perforating at least one hole in a membrane comprises the use of Inductively Coupled Plasma (ICP). In another embodiment, the step of perforating at least one hole in a membrane comprises the use of electron beam lithography followed by Inductively Coupled Plasma. According to some embodiments perforating at least one hole in a membrane comprises the use of any known method used for machining and/or milling.

In some embodiments, the step of coating the surface of the perforated membrane comprises the oxidation of a perforated silicon membrane.

In other embodiments, the step of coating the surface of the perforated membrane comprises the nitridation of a silicon dioxide membrane.

In certain embodiments, the etching of the membrane or the etching of the coating of the membrane comprises a chemical etching, a physical etching or a combination thereof. Each possibility represents a separate embodiment of the present invention.

In various embodiments, the chemical etching comprises the use of an acid reagent selected from phosphoric acid and hydrofluoric acid. Each possibility represents a separate embodiment of the present invention. In other embodiments, the chemical etching comprises the use of a base selected from potassium hydroxide and sodium hydroxide. Each possibility represents a separate embodiment of the present invention.

It will be recognized by one of skill in the art that the reagent for chemical etching is selected according to the substance to be etched. For example, phosphoric acid is suitable for etching silicon nitride, hydrofluoric acid is suitable for etching silicon dioxide and potassium hydroxide is suitable for etching silicon.

In some embodiments, the physical etching comprises the use of Ar, Xe or O₂ plasma. Each possibility represents a separate embodiment of the present invention.

In other embodiments, the combination of physical and chemical etching comprises the use of SF₆, CHF₃ or C₄F₈ plasma. Each possibility represents a separate embodiment of the present invention. It will be recognized by one of skill in the art that the plasma used for physical etching or the combination of physical and chemical etching is selected according to the substance to be etched.

In certain embodiments, the method of the present invention can be applied for the fabrication of nanoinjectors, nanoelectromechanical systems (NEMS) or microelectromechanical systems (MEMS). Each possibility represents a separate embodiment of the present invention.

In other embodiments, the aspect ratio of the obtained device is between about 1:1 and about 1:100.

According to another aspect, the present invention provides a device comprising at least one hollow nanoneedle, wherein the nanoneedle is supported by a membrane having two opposing surfaces and wherein the opening or aperture in the nanoneedle traverses the membrane. In one embodiment, the device is used for delivering molecules into cells. In another embodiment, the device comprises a plurality of hollow nanoneedles deployed in a predetermined array. In some embodiments, the device is fabricated according to the method of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 is a schematic representation of a molecular delivery system.

FIG. 2 is a schematic representation of the fabrication method of the present invention.

FIG. 3 is a scanning electron micrograph of silicon pillars which were formed using oxidization, optical lithography, masking and etching followed by milling using FIB to form nano-holes.

FIGS. 4A-4B are scanning electron micrographs of typical pillars obtained by using ring-shaped masks and e-beam lithography. The holes do not penetrate the silicon membrane. (4A) top view; and (4B) side view.

FIG. 5 is a schematic representation of a fabrication process of a nanoneedle array prepared from a Silicon On Insulator (SOI) wafer.

FIGS. 6A-6C are scanning electron micrographs of a typical nanoneedle array produced by the method of the present invention. Magnifications: (6A) ×1200; (6B) ×10,000; and (6C) ×25,000.

FIG. 7 is a confocal image showing the transfer of fluorescin through a nanoneedle array produced by the method of the present invention.

FIG. 8 is a confocal image of GFP and NIH3T3 cells which were grown on the nanoneedle array produced by the method of the present invention.

FIGS. 9A-9C are scanning electron micrographs of cells which were grown on a typical nanoneedle array produced by the method of the present invention. Magnifications: (9A) ×600; (9B) ×6,500; and (9C) ×15,000.

FIG. 10 is a scanning electron micrograph of a hollow structure having a rectangular shape which was produced by the method of the present invention. Magnification: ×3,500.

### DETAILED DESCRIPTION OF THE INVENTION

The present invention provides a method for fabricating a device which comprises at least one, preferably an array comprising a plurality of hollow nanoneedles of desired sizes and shapes on a supporting membrane. The hollow interior of the nanoneedles penetrates the supporting membrane and spans the membrane from one side to the other. The device may be suitable for use in a variety of applications including, in particular, as a molecular delivery system.

The method of the present invention overcomes the problems of the prior art thus providing a highly ordered nanoneedle array, wherein the nanoneedles are substantially aligned perpendicular to a supporting membrane. The method of the present invention allows the combination of nanoneedles having different sizes and shapes in a single device. The method of the present invention may use biocompatible materials thus affording molecular delivery systems which are particularly suitable in the fields of nanomedicine and nanodiagnostics. In addition, the method of the present invention may be applied in the fields of nanolithography and nanofluidics and for the integration of devices into lab-on-a-chip systems.

The present invention thus provides a method for fabricating a device which comprises at least one hollow nanoneedle which is supported by a membrane. In one embodiment, the device comprises one hollow nanoneedle. In another embodiment, the device comprises an array of hollow nanoneedles comprising a plurality of nanoneedles, for example between about 10,000 and about 1,000,000 nanoneedles. The hollow interior of the nanoneedles extends through the membrane. The method comprises the perforation of a membrane to form at least one hole that spans the membrane from one side to the opposite side thus forming a connection between the two sides of the membrane. The surface of the perforated membrane is then coated followed by the removal of the coated surface from one side of the membrane and the selective etching of a portion of the membrane from the same side of the membrane to expose the hollow pillars. It will be recognized by one of skill in the art that the surface of the perforated membrane can be coated so that one of the opposing surfaces of the membrane remains uncoated thus obviating the need for the removal of the coating from said surface.

For a better understanding of the invention and to show how it may be carried into effect, reference will now be made, purely by way of example, to the accompanying drawings. With specific reference now to the drawings in detail, it is stressed that the particulars shown are by way of example and for purposes of illustrative discussion of the preferred embodiments of the present invention only, and are presented with the purpose of providing what is believed to be the most useful and readily understood description of the principles and conceptual aspects of the invention. In this regard, no attempt is made to show structural details of the invention in more detail than is necessary for a fundamental understanding of the invention; the description taken with the drawings making apparent to those skilled in the art how the several forms of the invention may be embodied in practice.

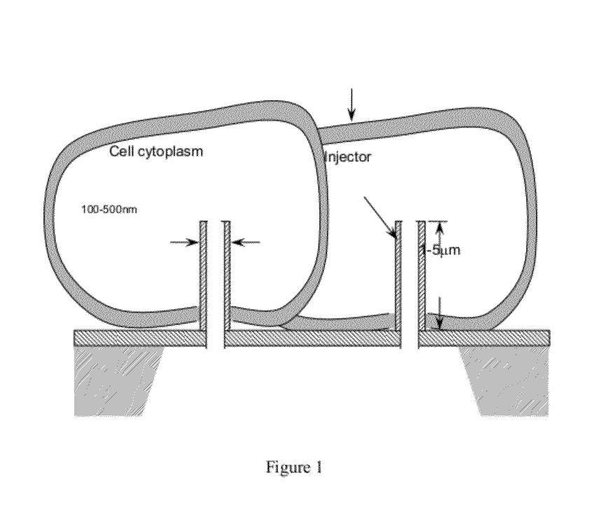

FIG. 1 shows a schematic illustration which demonstrates a molecular delivery system which comprises an array of nanoneedles that are located on a single supporting membrane. The hollow interior of the nanoneedles traverses the membrane thus allowing a passage of molecules from a reservoir located at the back side of the supporting membrane to cells which are cultured on the nanoneedle array. The nanoneedles penetrate the cells thus connecting the reservoir directly to the cytoplasms. This allows a direct communication with the cell interior which can be used for delivering molecules to the cells and probing cell responses.

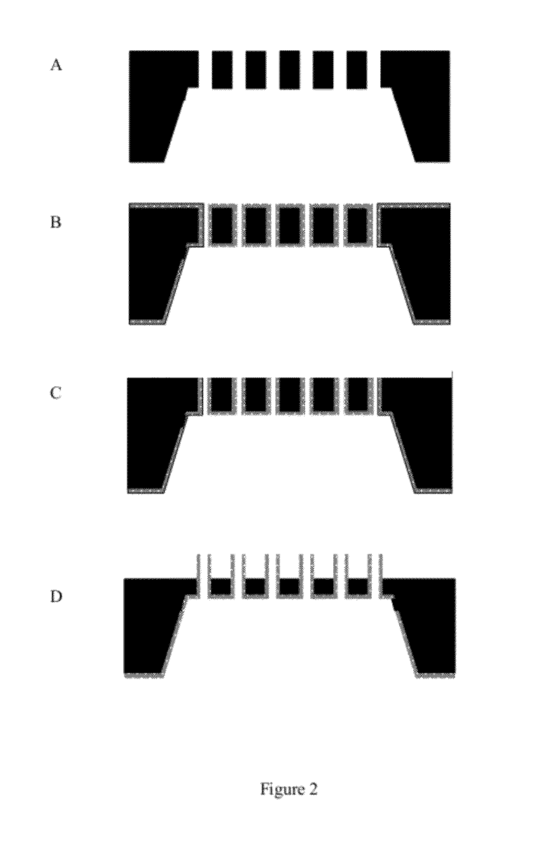

FIG. 2 is a schematic representation which demonstrates one non-limiting manner in which the method of the present invention can be implemented. In this example, the process utilizes a membrane in which sub-micron holes that pass through the membrane are defined and drilled, leaving a perforated membrane (panel A). It will be recognized by one of skill in the art that the definition of holes using e.g. e-beam lithography is optional. The membrane can be selected from various materials including, but not limited to, metalloids, metals, organic polymers, and the like. Suitable membranes include, but are not limited to, membranes comprising silicon (e.g. silicon wafers, membranes comprising silicon dioxide, membranes comprising silicon nitride and combinations thereof), membranes comprising germanium (e.g. germanium membranes, germanium dioxide membranes, germanium nitride membranes and combinations thereof), membranes comprising aluminum or alumina and combinations thereof, membranes comprising boron nitride and the like. Membrane perforation can be performed as is known in the art. Suitable techniques for membrane perforation include, but are not limited to, plasma (e.g. Inductively Coupled Plasma—ICP, Reactive Ion Etching—RIE) and Focused Ion Beam (FIB). Each possibility represents a separate embodiment of the present invention. For example, membrane perforation can be performed using the Bosch process as is known in the art which comprises alternating cycles of etching and deposition using e.g. a Deep Reactive Ion Etching (DRIE) machine or Inductively Coupled Plasma (ICP). Combinations of several techniques can also be applied. Then, the surface of the perforated membrane is coated (panel B). The coating is selected so as to provide the subsequent selective etching of the membrane and/or coating. Suitable examples for coating include, but are not limited to, coating of a silicon or germanium membrane with silicon dioxide or germanium dioxide; coating of a silicon dioxide or germanium dioxide membrane with silicon nitride or germanium nitride; coating of an aluminum membrane with alumina etc. In one embodiment, the coating of a silicon membrane comprises the oxidation of a silicon membrane so as to obtain a silicon dioxide coating. The thickness of the coating can be determined according to need through controlling the time and temperatures at which the oxidation step is performed as is known in the art. For example, oxidation of bare silicon for about 3 hours in 1000° C. will result in a coating of about 100 nm. When coating is performed by oxidizing the membrane, the coating is advantageously centered with respect to the holes. The process then comprises the removal of the coating from one side of the membrane (panel C) followed by the partial selective etching of the membrane from the same side in order to turn the once buried holes into hollow pillars (needles), sticking out several microns above the surface (panel D). The removal of the coating can be performed, for example, using chemical etching, physical etching or a combination thereof. Each possibility represents a separate embodiment of the present invention. Suitable reagents for chemical etching include, but are not limited to, acids such as phosphoric acid or hydrofluoric acid; bases such as potassium hydroxide or sodium hydroxide and the like. The etching reagent can be selected according to the substance to be etched. For example, phosphoric acid is suitable for etching silicon nitride, hydrofluoric acid is suitable for etching silicon dioxide and potassium hydroxide is suitable for etching silicon. Suitable physical etching includes, but is not limited to, the use of plasma based on e.g. Ar, Xe or O₂. Suitable physical and chemical etching includes, but is not limited to, the use of plasma based on e.g. SF₆, CHF₃ or C₄F₈. It is contemplated that the plasma is selected according to the substance to be etched.

The method of the present invention provides devices with high aspect ratios between about 1:1 and about 1:100, suitable as nanoinjectors, nanoelectromechanical systems (NEMS) or microelectromechanical systems (MEMS). Each possibility represents a separate embodiment of the present invention. In one embodiment, the method of the present invention provides a molecular delivery device suitable for delivering molecules into cells.

In some embodiments, the step of perforating the membrane comprises the use of physical perforation, chemical perforation or any combination thereof as is known in the art. Suitable physical perforation comprises:
The creation of a perforated membrane using molds with pillars, to which a thin membrane (e.g. a polymer membrane) is applied using e.g., spin coating.
FIB patterning which involves the use of an energetic beam of gallium ions focused at one point of the specimen, thus affording perforation of said point.
Plasma etching which involves the use of a non-focused flux of energetic ions to remove materials from one point of the specimen. This technique usually requires a mask to shield the non-perforated regions. The mask can be selected according to its physical stiffness (if chemically inert plasma is used). Alternatively, the mask may be selected according to its chemical selectivity thus affording chemically reactive plasma which provides both physical and chemical etching.
Radiation which involves the use of nuclear reactors to create nanopores or the use of laser ablation using a focused laser. Alternatively, photolithography using a photoresist can also be applied.
   Suitable chemical perforation comprises:
Etching (e.g. using a strong acid or base) with a mask layer to protect points which are not subjected to perforation.
Alternating electrical currents to a medium in which a chemical reaction which results in the creation of pores is performed. For example, porous silicon can be fabricated by HF etching of a silicon wafer under AC currents, without masks.

In various embodiments, the step of coating the membrane comprises modifying the surface of the perforated membrane thereby forming a coated perforated membrane. In some embodiments, the modification comprises the oxidation of the membrane. In other embodiments, the step of coating the membrane comprises the use of the perforated membrane as a support onto which a different substance is applied as a vapor or a liquid. Suitable techniques include, but are not limited to, vapor phase epitaxy (e.g. physical vapor deposition or chemical vapor deposition), liquid phase epitaxy, solid phase epitaxy, spin coating, dip-coating, screen printing, so-gel printing and the like. Each possibility represents a separate embodiment of the present invention. Chemical Vapor Deposition (CVD) comprises the use of a vapor phase (usually diluted with an inert gas carrier), which chemically reacts on the surface of a substrate to deposit a solid film. Various CVD techniques include, but are not limited to, atmospheric pressure CVD, low pressure CVD/very low pressure CVD, metaloorganic CVD, and plasma enhanced CVD. Physical Vapor Deposition (PVD) comprises:
thermal evaporation using a hot material in vacuum.
sputtering using inert plasma which is sputtered on the membrane.
molecular beam epitaxy using atomic streams that contain the coating material. The membrane is heated and placed in high vacuum where the atomic streams impinge on its surface. This method provides coating layers with thicknesses in the range of up to a few nanometers.
laser ablation deposition using an intense laser radiation to erode a target and deposit the eroded material on the membrane. This technique is useful for thin coating.
   Non-epitaxial coating methods comprise:
spin coating, wherein the coating material is applied on the substrate which is then rotated on a spinning wheel at high speed so as to allow excess material to accumulate on the edges by centrifugal forces. This method provides a uniform coating layer.
dip coating, wherein the membrane is dipped in a solution containing the coating material which assembles on the surface of the membrane.
Casting, wherein a paste containing the coating material and a solvent is applied on the membrane. The solvent than evaporates leaving a coating layer on the surface of the membrane.
Inkjet printing/Screen printing using nozzles or metal masks through which the coating paste is pushed onto the membrane at selective places.
Glow Discharge Polymerization, wherein the membrane is coated with a monomer (usually by spraying, spin coating, or dip coating) and then the coated membrane is exposed to plasma, which induces polymerization.

In certain embodiments, the step of selectively etching a portion of the membrane comprises chemical etching, physical etching or a combination of physical and chemical etching. Thus, according to the principles of the present invention any pairs of materials (coating and membrane) that can be etched selectively due to differences in the rate of etching or by using different etching reagents can be used when applying the method disclosed herein. Selective etching can be performed by applying an etching process wherein the rate of etching is significantly different between the coating and membrane. The rate of etching can be determined either by mass transport or by the kinetics of the chemical reactions involved in the etch process. Common etching techniques include, but are not limited to, dry etching (using mainly plasma and gas-phase etchants) and wet etching (using solutions of chemically active species—usually strong acids or bases). Selective etching can also be performed by directionality wherein certain directions are more susceptible to the etching process than other directions. In wet etching, only single crystals can be etched with high directionality.

Dry etching techniques comprise:
Ion Beam Milling, Physical Sputtering using a focused ion beam (e.g., FIB) or an unfocused ion beam (e.g., plasma, ion beam sputtering). The technique involves the use of highly energetic ions which hit the surface to be etched and subsequently sputter away material from the surface. Selectivity can be achieved by exposing only parts of the membrane to the etching process (e.g. by focusing the ion beam on selected areas of the membrane).
Reactive Ion Etching (RIE) using highly energetic ions which are also chemically active. The ions hit the surface to be etched and induce a chemical reaction on the surface thereby removing volatile gases. This process is more selective and allows the combination of physical and chemical etching. Alternatively, the process can be purely chemical wherein the plasma serves as a means to bring the reactants close to the surface of the membrane.
Plasma Etching, Deep RIE (DRIE) combined with Inductively Coupled Plasma (ICP). This technique provides a faster and more selective etching due to the use of much stronger plasma.
Vapor Phase Etching, wherein the membrane to be etched is exposed to a vapor of chemically active species (for example, silicon can be etched using XeF₂ vapor).

Wet etching comprises the immersion of the membrane in a solution of a chemically active reagent as described herein above. The etching is purely chemical, and it usually relies on the difference in the rate of etching of different crystallographic planes of the material. Variations of wet etching are usually in the composition of the etchant solution (which depends on the material to be etched), the etching conditions (e.g. temperature), and the method used to terminate the etching process (e.g. the introduction of an etch ‘stop layer’ which is etched much slower than the remainings of the membrane. Suitable stop layer includes, but is not limited to, a different material (e.g., SiO₂ is a stop layer when using KOH to etch silicon), a different doping (e.g., silicon layers which are heavily doped usually etch much slower than undoped layers). Selective wet etching can also be performed by directionality wherein certain crystallographic planes can serve as etch stop layers.

The principles of the invention are demonstrated by means of the following non-limiting examples.

### COMPARATIVE EXAMPLE 1

### Attempts to Fabricate Hollow Si Nanoneedles on a Supporting Membrane

Attempts to fabricate solid nanopillars supported by a membrane and subsequently to mill holes within the nanostructures as well as attempts to fabricate hollow nanoneedles with holes that traverse the supporting membrane by making ring-shaped masks using e-beam lithography and subsequently etching the membrane in areas which are not protected by the ring shaped masks were unsuccessful.

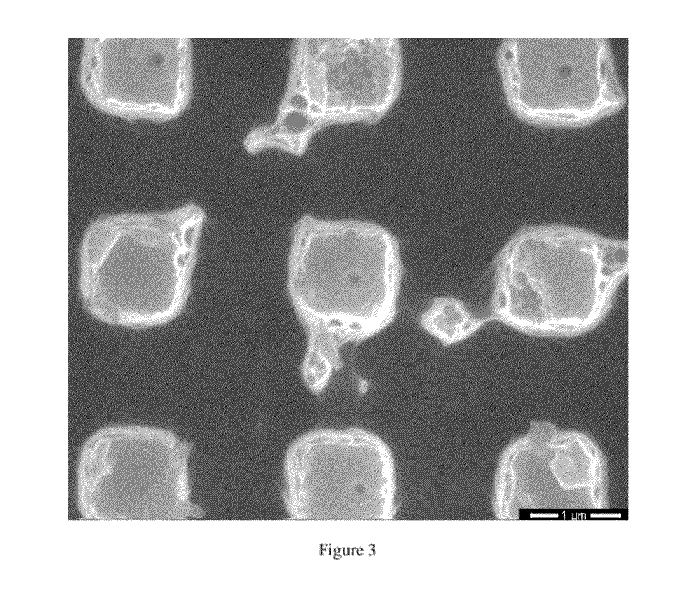

Solid silicon nanopillars were fabricated using optical lithography and DRIE etching. Attempts were made to mill holes through these nanopillars and the membrane that supports them, using a FIB machine (model Strata-400S Dueal Beam FIB, manufactured by FEI company; FIG. 3). The attempts were carried out using two stages. First, a high-aspect-ratio (˜1:80) hole was milled in a flat silicon wafer. Next, milling of non-flat surface containing a nanopillar was addressed.

Holes possessing an aspect ratio of 1:80 were milled in a flat silicon surface. Each hole was 12 microns deep, with nearly vertical walls from 1 micron below the surface (where the hole diameter is about 50 nm) to the hole bottom. At the uppermost micron, closest to the surface, the hole profile was not vertical but a funnel formed, making the opening 250 nm in diameter. In order to achieve a high aspect ratio, milling was performed by a series of rapid pulses rather than a single, long pulse of the ion beam. The current of the ion beam was set to 48 pA and the milling interval was about several minutes per hole.

This method of milling holes inside nanopillars where the hole is positioned at the center of the nanopillars was unsuccessful for the following reasons: First, when the pillars are of submicrometric diameters, it was very hard to align the ion beam to the center of a nanopillar. Electrical charges accumulated on non-flat surfaces made from an electrically non-conducting material (e.g. silicon dioxide), thereby deflecting the ion beam and affecting its stability. This effect (termed “charging”) was more pronounced when more than a single hole was milled in parallel. It was found that when the ion beam is set down from the milling point before the hole is milled to its entire depth, it is practically impossible to align the beam to the same point again. Alternatively, if a single point was milled, drifts in the machine made it impossible to automatically align the machine at the precise location for the milling of another hole, and a manual intervention was required after each milling. In addition, if the milling angle was not perfectly aligned, instead of milling a hole that perforates the supporting membrane the hole would perforate the side wall of the pillar, or simply would not penetrate the membrane. Furthermore, funnels were formed at the uppermost part of the holes, thereby thinning the top of the nanopillar walls at these locations. The nanopillars were often entirely consumed. Thus, using conventional techniques such as FIB milling to mill holes positioned at the center of each nanopillar in an array of nanopillars was not feasible.

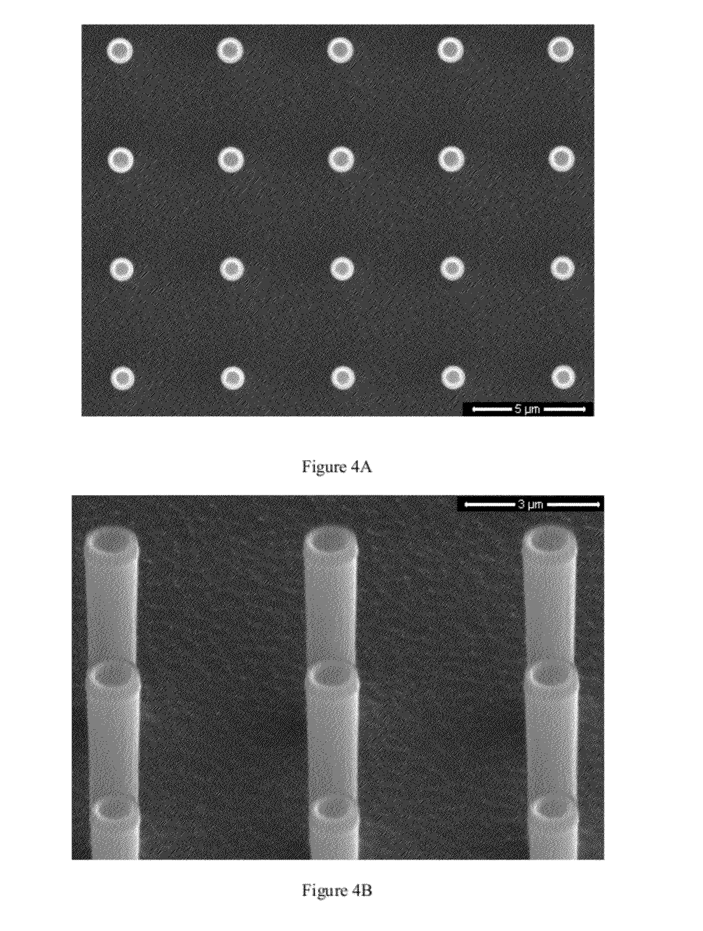

Other attempts have been made to obtain hollow nanoneedles with holes that traverse the supporting membrane. The attempts were performed by making ring-shaped masks using e-beam lithography and subsequently etching the membrane in areas which are not protected by the ring shaped masks. Due to the geometry of the capillaries, plasma etching provides differences in the rate of etching inside the capillaries (higher rate) as compared to the bulk. Thus, the hole inside the capillaries is deeper as compared to the exterior of the capillaries. FIGS. 4A and 4B demonstrate pillars that were obtained by this method. The holes inside the pillars do not penetrate the membrane as is evident from the top view (FIG. 4A).

### EXAMPLE 2

### Fabrication of Hollow SiO₂ Nanoneedles on a Silicon Membrane

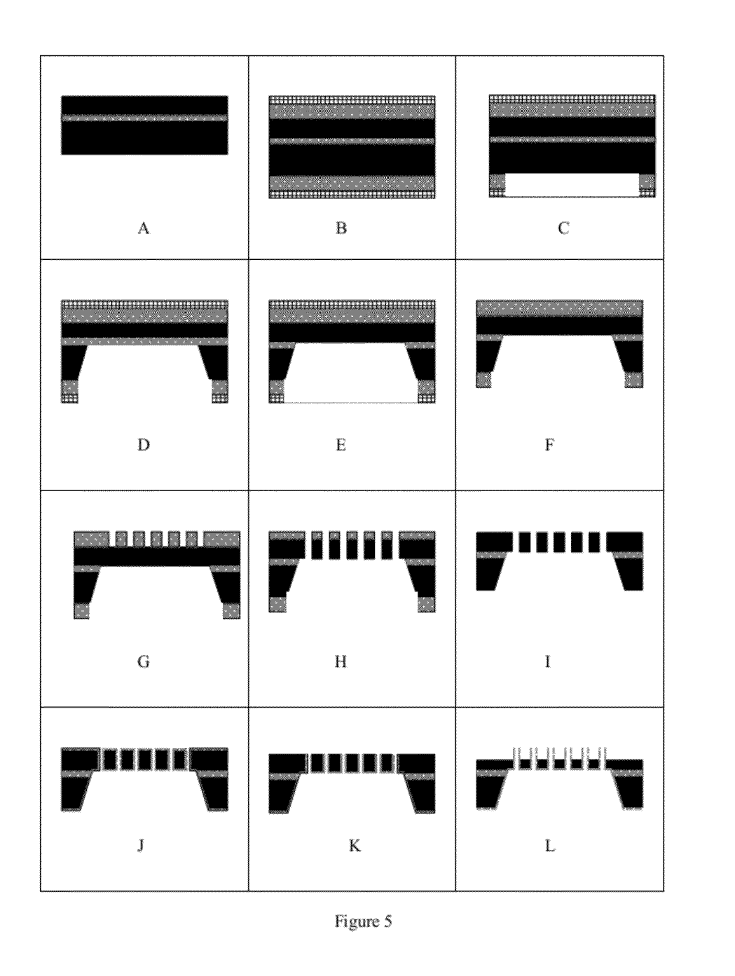

The fabrication of hollow SiO₂ nanoneedles on a Silicon On Insulator (SOI) wafer was performed. The process is schematically shown in FIG. 5. The thickness of the obtained membrane can be controlled by using different SOIs with varying thicknesses. An SOI wafer (purchased from Virginia Semiconductors, USA) was obtained (FIG. 5A). The SOI wafer was thermally oxidized (gray dotted pattern) and nitrified (grid pattern) (FIG. 5B). Devices containing thin silicon membranes were then fabricated as follows. Optical lithography was used to define the outline of the chips and membranes and to set the directionality of the wafer. The handle side of the wafer was then patterned by optical lithography (FIG. 5C) followed by etching in 33% KOH solution, with the nitride layer (grid pattern) serving as a mask and the middle oxide layer (gray dotted pattern) serving as a stop layer (FIG. 5D). After the formation of a thin, suspended, membrane, the middle oxide layer was removed by dipping the wafer in HF at 25° C. (FIG. 5E). The nitride layer was subsequently removed by dipping the wafer in H₃PO₄ at 175° C. (FIG. 5F). Next, the perforation of the membrane was performed. The membrane was spin coated with a positive electron-beam resist. E-beam lithography was used to define sub-micrometric patterns of desired shape and size (FIG. 5G). Inductively Coupled Plasma (ICP) was then used to perforate the membrane, using alternating etching and protecting cycles. The resulting perforated membrane (FIG. 5H) was cleaned from the remaining of the oxide layer by dipping of the upper side in HF (FIG. 5I), followed by cleaning with a standard RCA cleaning process before being thermally oxidized for 185 minutes at a temperature of 1000° C. (FIG. 5J). This step yielded about 100 nm thick oxide layer. The oxide layer grew uniformly on the surface of the membrane, narrowing the holes during the process. Typical holes are about 400 nm wide and the external diameter of the pillars is about 600 nm. Subsequently, the oxide layer on top of the membrane was removed using Reactive Ion Etching (RIE) with CHF₃ plasma (FIG. 5K), and the exposed silicon layer was thinned by a Deep Reactive Ion Etching process. The thinning process, being 60 times faster with silicon compared to silicon dioxide, reveals the buried SiO₂ nanoneedles. Thus, the once buried silicon holes turned into hollow silica pillars (needles), sticking out several microns above the surface (FIG. 5L).

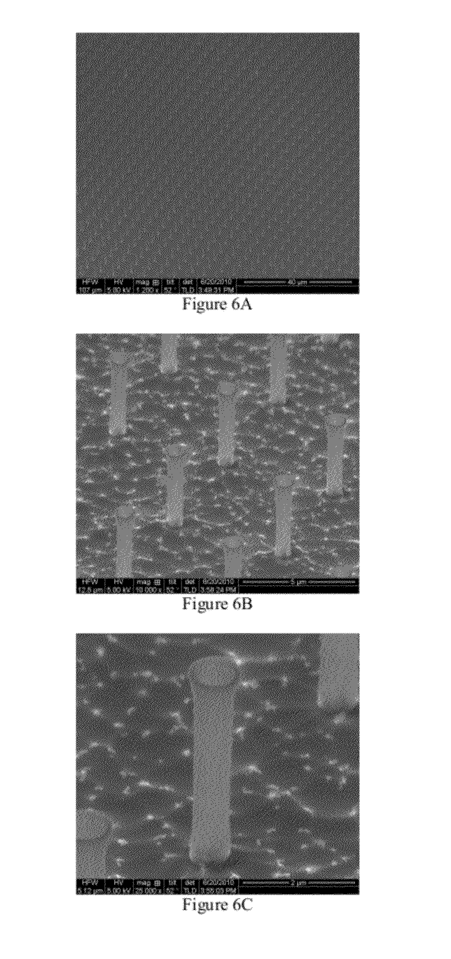

Typical nanoneedles, fabricated by the aforementioned process, are shown in FIG. 6. The nanoneedles array (FIG. 6A) spans 300 μm×300 μm, with nanoneedles situated every 5 μm (FIG. 6B). Each nanoneedle (FIG. 6C) is about 6 μm high and has a 0.5 μm external diameter. The void of the nanoneedle traverses additional 5 μm of the supporting silicon membrane. The inner diameter of the needle shown in the figure is roughly 200 nm.

### EXAMPLE 3

### Molecular Delivery System

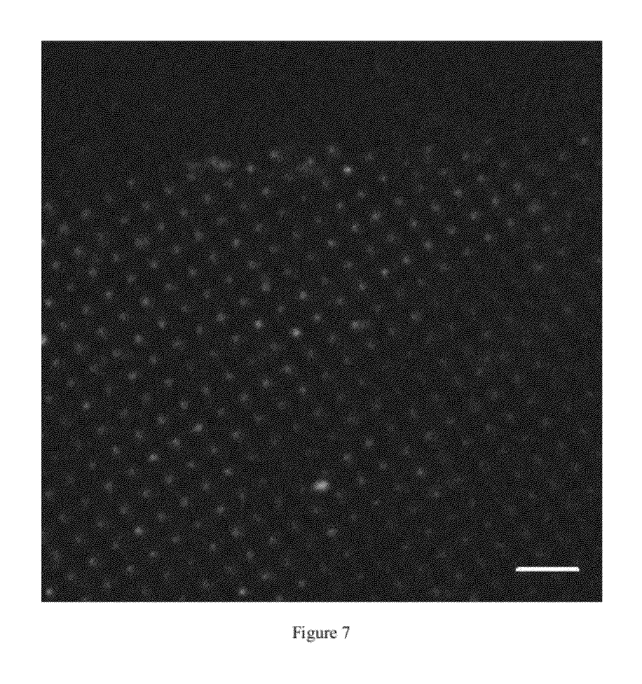

In order to verify that the holes traverse the entire length of the needles, Fluorescin (Sigma Aldrich, Rehovot, Israel) was transferred through the needles (FIG. 7). The time evolution of the fluorescence signal obtained using a confocal microscope when introducing 10 μM Fluorescin solution in PBS to the reservoir at the rear side of the needles and PBS solution to the front side, was measured. A calibration of the microscope showed that signals resulting from concentrations as low as 100 nM of Fluorescin were detectable in this manner. The time evolution showed a buildup of the signal which reached a plateau after about 80 minutes. Without being bound by any theory or mechanism of action, the observed buildup of the signal is mainly attributed to the flux of the molecules through the needles, which is the rate limiting step until a sufficient amount of molecules transfers through the needle to afford a detectable signal in the cells.

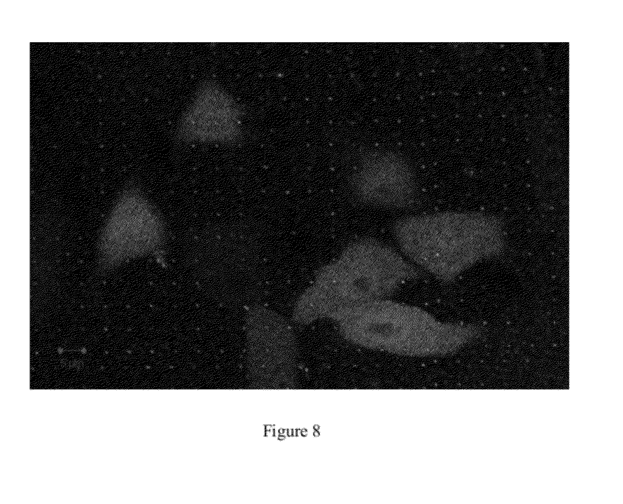

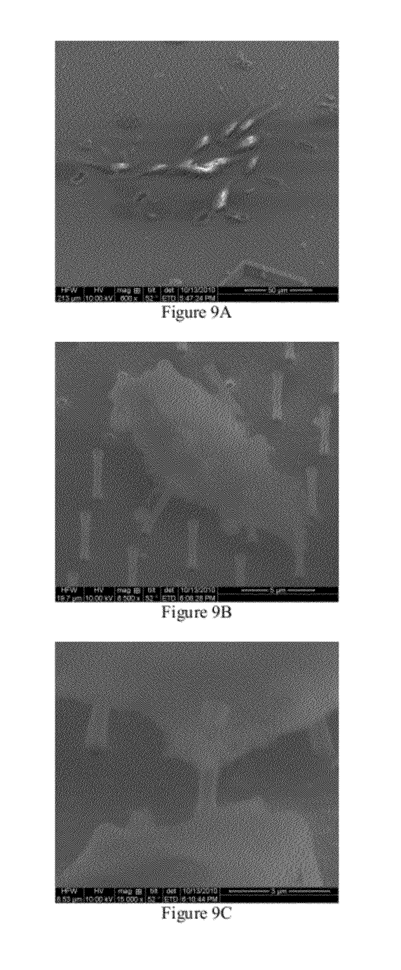

In order to test the functionality of the needle array as a molecular delivery system, NIH-3T3+GFP mouse fibroblast cells were cultured on the arrays. The cells, as seen in FIG. 8, were able to adhere, spread, proliferate and form colonies on the arrays. In order to better examine the interaction between the cells and the nanoneedles, the cells were fixated on the arrays and observed using SEM. For cell fixation the following procedure was used (all reagents were purchased from Bar-Naor, Israel): cells were washed twice in PBS, and then fixed with 2% glutaraldehyde solution for 30 minutes. This was followed by 3 washes in cacodylate buffer at pH 7.4; in order to improve the contrast, cells were fixed with osmium tetroxide, gradually transferred to ethanol, and Critically Point Dried. Finally, the specimens were sputter-coated with Chromium and observed. FIG. 9 shows typical cells. It is evident that the cells were able to spread on the nanoneedles and form fillopodia around the needles. The top of the needles is situated at the same plane as the cytoskeleton. Some nanoneedles, especially those close to the cell's periphery, are slightly bent towards the cell body. Without being bound by any theory or mechanism of action, indentation might be attributed to forces applied by vital cells.

### EXAMPLE 4

### Fabrication of Hollow SiO₂ Structures of Different Shapes on a Silicon Membrane

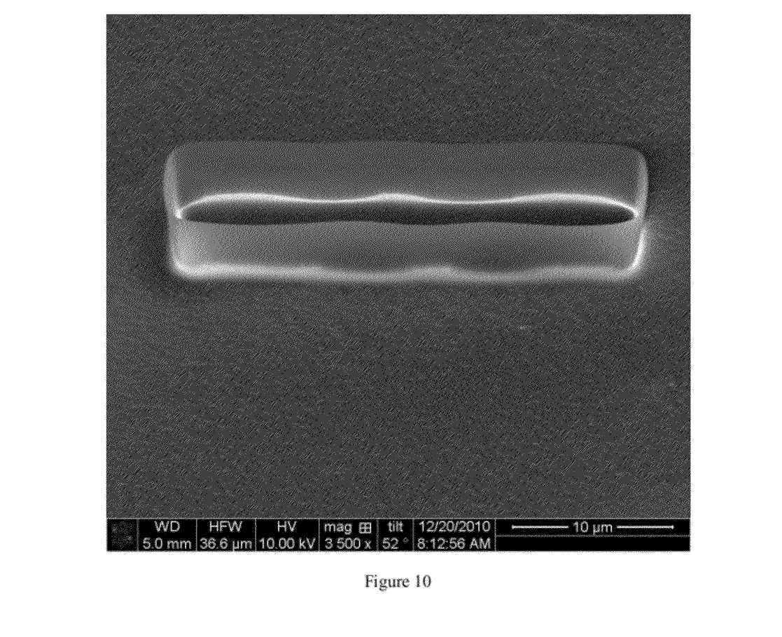

FIG. 10 demonstrates a different morphology of a device prepared in accordance with the method of the present invention. The device was prepared as described in Example 2 hereinabove. FIG. 10 shows a relatively large hollow rectangle having the same wall width and height above the surface as the nanoneedles prepared in the same manner.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

1. A method of fabricating a device comprising at least one hollow nanoneedle wherein the nanoneedle is supported by a membrane having two opposing surfaces and wherein the aperture in the nanoneedle traverses the membrane, the method comprising the steps of:
a. perforating a membrane to form at least one hole that traverses the membrane from one side to the opposite side;
b. coating the surfaces of the perforated membrane obtained in step (a);
c. removing the coated surface obtained in step (b) from one side of the membrane; and
d. selectively etching a portion of the membrane from said one side of the membrane.

2. The method according to claim 1, wherein the membrane is selected from the group consisting of silicon, silicon dioxide, silicon nitride, germanium, germanium dioxide, germanium nitride, aluminum, alumina, boron nitride and combinations thereof.

3. The method according to claim 2 wherein the membrane is selected from the group consisting of silicon, silicon dioxide and silicon nitride and combinations thereof.

4. The method according to claim 2, wherein the membrane comprises silicon.

5. The method according to claim 1, wherein the coating of the surface of the membrane is selected from the group consisting of silicon dioxide, silicon nitride, germanium dioxide, germanium nitride, and alumina.

6. The method according to claim 4, wherein the coating of the surface of the membrane is selected from the group consisting of silicon dioxide and silicon nitride.

7. The method according to claim 1, wherein the step of perforating a membrane to form at least one hole comprises the use of at least one technique selected from plasma and Focused Ion Beam (FIB).

8. The method according to claim 6, wherein the step of perforating a membrane to form at least one hole comprises the use of Inductively Coupled Plasma (ICP).

9. The method according to claim 1, wherein the step of perforating a membrane to form at least one hole comprises the use of electron beam lithography followed by Inductively Coupled Plasma (ICP).

10. The method according to claim 1, wherein the step of coating the surface of a perforated membrane comprises the oxidation of the perforated silicon membrane.

11. The method according to claim 1, wherein the step of coating the surface of a perforated membrane comprises the nitridation of the perforated silicon dioxide membrane.

12. The method according to claim 1, wherein the step of removing the coated surface of the membrane comprises a chemical etching, a physical etching or a combination thereof.

13. The method according to claim 1, wherein the step of selectively etching the membrane comprises a chemical etching, a physical etching or a combination thereof.

14. The method according to claim 12, wherein the chemical etching comprises the use of an acid reagent selected from phosphoric acid and hydrofluoric acid.

15. The method according to claim 12, wherein the chemical etching comprises the use of a base reagent selected from potassium hydroxide and sodium hydroxide.

16. The method according to claim 12, wherein the physical etching comprises the use of Ar, Xe or O₂ plasma.

17. The method according to claim 12, wherein the combination of physical and chemical etching comprises the use of SF₆, CHF₃ or C₄F₈ plasma.

18. The method according to claim 1, wherein the aspect ratio of the device is between about 1:1 and about 1:100.

19. A device for delivering molecules into cells, wherein the device comprises at least one hollow nanoneedle wherein the nanoneedle is supported by a membrane having two opposing surfaces and wherein the aperture in the nanoneedle traverses the membrane.

20. The device for delivering molecules into cells according to claim 19, wherein the device is fabricated according to claim 1.

21. The device of claim 19 comprising a plurality of hollow nanoneedles deployed in a predetermined array.
